(19) United States
(12) Patent Application Publication
(10) Pub. No.: US 2014/0024663 A1
(43) Pub. Date: Jan. 23, 2014

(54) ATB(0,+) AMINO ACID TRANSPORTER AS A DRUG TARGET FOR TREATMENT OF ESTROGEN RECEPTOR-POSITIVE BREAST CANCER

(71) Applicant: GEORGIA HEALTH SCIENCES UNIVERSITY RESEARCH INSTITUTE, INC., AUGUSTA, GA (US)

(72) Inventors: VADIVEL GANAPATHY, MARTINEZ, GA (US); MUTHUSAMY THANGARAJU, EVANS, GA (US)

(21) Appl. No.: 13/940,507

(22) Filed: Jul. 12, 2013

Related U.S. Application Data
(60) Provisional application No. 61/672,403, filed on Jul. 17, 2012.

(51) Int. Cl. A61K 31/405; A61K 45/06; A61K 31/52

(52) U.S. Cl. CPC A61K 31/405; A61K 31/52; A61K 45/06
USPC 514/263.4; 514/419

(57) ABSTRACT

The present invention includes inhibitors of the amino acid transporter ATB^{0,+} and methods of uses thereof for the treatment of estrogen receptor-positive breast cancer.

### CONTINUING APPLICATION DATA

This application claims the benefit of U.S. Provisional Application Ser. No. 61/672,403, filed Jul. 17, 2013, which is incorporated by reference herein in its entirety.

### GOVERNMENT FUNDING

This invention was made with government support under GM065344 and CA152396 awarded by the National Institutes for Health. The Government has certain rights in the invention.

### BACKGROUND

Cancer is a widespread and deadly disease. Although a variety of therapeutic strategies are currently used for treatment of cancer, for many cancers these treatments do not offer a permanent cure for the disease. Significant improvements in the treatment of cancer have proven difficult to develop. Currently, the standard to measure the success of a new anti-cancer drug is often an increase in the survival of cancer patients in terms of months, not in years. There is a need for improved agents for the treatment of cancer.

### SUMMARY OF THE INVENTION

The present invention includes methods of treating estrogen receptor positive breast cancer in a subject, the method including administering to the subject a composition including an inhibitor of the ATB^{0,+} amino acid transporter. In some aspects, the inhibitor of the ATB^{0,+} amino acid transporter is administered in an amount effective to inhibit amino acid transport into cancer cells. In some aspects, the cancer cells demonstrate enhanced expression of the ATB^{0,+} amino acid transporter. In some aspects, the estrogen receptor positive breast cancer is metastatic.

The present invention includes methods of killing estrogen receptor positive breast cancer cells, the method including contacting the cells with a composition including an inhibitor of the ATB^{0,+} amino acid transporter. In some aspects, the inhibitor of the ATB^{0,+} amino acid transporter is administered in an amount effective to inhibit amino acid transport into cancer cells. In some aspects, the cancer cells demonstrate enhanced expression of the ATB^{0,+} amino acid transporter. In some aspects, the estrogen receptor positive breast cancer is metastatic.

The present invention includes methods of inducing amino acid deprivation and/or autophagy in a cancer cell, the method including contacting the cancer cell with an inhibitor of the ATB^{0,+} amino acid transporter. In some aspects, amino acid transport into cells is inhibited. In some aspects, the cancer is a breast cancer. In some aspects, the breast cancer is an estrogen receptor positive breast cancer. In some aspects, the cancer is metastatic.

The present invention includes methods of inducing apoptosis in a cancer cell, the method including contacting the cancer cell with an inhibitor of the ATB^{0,+} amino acid transporter. In some aspects, the cancer is a breast cancer. In some aspects, the breast cancer is an estrogen receptor positive breast cancer. In some aspects, the cancer is metastatic.

The present invention includes methods of inducing apoptosis in a cancer cell, the method including contacting the cancer cell with an inhibitor of the ATB^{0,+} amino acid transporter and an inhibitor of autophagy. In some aspects, the cancer is a breast cancer. In some aspects, the breast cancer is an estrogen receptor positive breast cancer. In some aspects, the cancer is metastatic.

In some aspects of the methods of the present invention, the inhibitor of the ATB0,+ amino acid transporter includes a tryptophan derivative.

In some aspects of the methods of the present invention, the inhibitor of the ATB0,+ amino acid transporter includes alpha methyl tryptophan.

In some aspects of the methods of the present invention, the composition includes the L isomer of alpha methyl tryptophan and does not includes the D isomer of alpha methyl tryptophan.

In some aspects of the methods of the present invention, the method further includes administration of an inhibitor of autophagy. In some aspects, an inhibitor of autophagy includes 3-methyladenine, suppressive miR-101, lucanthone, chloroquine, hydroxychloroquine, N-acetyl-L-cysteine, L-asparagine, bafilomycin A1 from Streptomyces griseus, catalase, JRF 12 N2,N4-dibenzylquinazoline-2,4-diamine (DbeQ), (2S,3S)-trans-Epoxysuccinyl-L-leucylamido-3-methylbutane ethyl ester EST (E-64d), leupeptin hemisulfate, 2-(4-Morpholinyl)-8-phenyl-1(4H)-benzopyran-4-one hydrochloride (LY-294,002), pepstatin A, thapsigargin, or wortmannin from Penicillium funiculosum.

In some aspects of a method of the present invention, the method further includes administration of an additional therapeutic agent.

In some aspects of the methods of the present invention, the composition is formulated for parenteral delivery. In some aspects, the composition is formulated for enteral delivery.

In some aspects any of the compositions and methods of the present invention, the inhibitor of the ATB^{0,+} amino acid transporter includes a tryptophan derivative. In some aspects, the composition may further include a pharmaceutically acceptable carrier.

In some aspects any of the compositions and methods of the present invention, the inhibitor of the ATB^{0,+} amino acid transporter includes alpha methyl tryptophan. In some aspects, the composition may further include a pharmaceutically acceptable carrier.

In some aspects any of the compositions and methods of the present invention, the composition includes the L isomer of alpha methyl tryptophan and does not includes the D isomer of alpha methyl tryptophan. In some aspects, the composition may further include a pharmaceutically acceptable carrier.

In some aspects any of the compositions and methods of the present invention, the inhibitor of the ATB^{0,+} amino acid transporter includes an alpha methyl tryptophan derivative. In some aspects, the composition may further include a pharmaceutically acceptable carrier.

The terms “comprises” and variations thereof do not have a limiting meaning where these terms appear in the description and claims.

Unless otherwise specified, “a,” “an,” “the,” and “at least one” are used interchangeably and mean one or more than one.

### BRIEF DESCRIPTION OF THE FIGURES

FIGS. 1A to 1C. Upregulation of SLC6A14 in ER-positive breast cancer. FIG. 1A is a RT-PCR analysis of SLC6A14 mRNA in primary human breast cancer tissues and corresponding adjacent normal tissues (N, normal; T, tumor). FIG. 1B is an immunohistochemical analysis of SLC6A14 protein in ER-positive breast cancer tissues and normal breast tissues. FIG. 1C is a RT-PCR analysis of SLC6A14 mRNA and ERα mRNA in human breast cancer cell lines (ER⁺ BC, ER-positive breast cancer cell lines; ER⁻ BC, ER-negative breast cancer cell lines).

FIGS. 2A-2C. Regulation of SLC6A14 expression by estrogen/ER. In FIG. 2A, ZR75.1 and BT474 cells were cultured for 5-6 passages in control medium (10% fetal bovine serum with phenol red) and phenol red-free medium (10% or 5% charcoal-stripped fetal bovine serum), and then the levels of ERa mRNA and SLC6A14 mRNA were determined by RT-PCR (P1, P2, P3, P4, P5, and P6 refer to passage numbers). In FIG. 2B, BT474 cells were treated with estradiol (10 nM) in the presence or absence of antiestrogens (1 μM) for 24 h, and then used for RT-PCR analysis of SLC6A14 mRNA and ERa mRNA (E2, estradiol; TAM, tamoxifen; 4-OH TAM, 4-hydroxytamoxifen; ICI 182780, FULVESTRANT™). In FIG. 2C, BT474 cells were transfected with either SLC6A14-promoter (˜3 kb)-luciferase construct or empty vector and then treated with estradiol (10 nM) in the presence and absence of antiestrogens (1 μM). Following the treatment, the reporter activity was measured. Data represent values after normalization with β-galactosidase activity for differences in transfection efficiency.

FIGS. 3A and 3B. Natactivation kinetics of SLC6A14-mediated transport of leucine (FIG. 3A) and arginine and glutamine (FIG. 3B). Human SLC6A14 was expressed heterologously in X. laevis oocytes by microinjection of cRNA. Four days following injection, oocytes were used for electrophysiological studies using the two-microelectrode voltage-clamp method. Uninjected oocytes showed negligible currents when perifused with leucine, arginine, or glutamine (1 mM). cRNA-injected oocytes showed marked inward currents when perifused with these three amino acids, and the magnitude of the currents increased with increasing concentrations of Na⁺ (concentration of was kept constant at 100 mM). Since the expression levels varied from oocyte to oocyte resulting in varying magnitudes of amino acid-induced currents, the currents were normalized by taking the magnitude of the currents induced at 100 mM Na⁺ as 1 in each oocyte and calculating the magnitude of the currents induced at other concentrations of Na⁺ as a fraction of this maximal current. The experiments were done with three different oocytes and the results are given as means±SE. Insets, Hill plots.

FIG. 4. Cl⁻-activation kinetics of SLC6A14-mediated transport of leucine, arginine, and glutamine. The experiments were done as described for the Na⁺-activation kinetics. cRNA-injected oocytes showed marked inward currents when perifused with these three amino acids, and the magnitude of the currents increased with increasing concentrations of Cl⁻ (concentration of Na⁺ was kept constant at 100 mM). The experiments were done with three different oocytes and the results are given as means±SE.

FIGS. 5A to 5C. Blockade of SLC6A14-mediated transport of leucine (FIG. 5A), glutamine (FIG. 5B), and arginine (FIG. 5C) by α-MT. Oocytes injected with human SLC6A14 cRNA were perifused with 100 M leucine, 100 μM glutamine, or 100 μM arginine under varying experimental conditions as indicated. Perifusion of the oocytes with the amino acids induced inward currents in the presence of NaCl, and the currents disappeared when NaCl was replaced with N-methyl-D-glucamine (NMDG) chloride. Re-perifusion of the same oocytes with the amino acids again induced inward currents in the presence of NaCl, but these currents disappeared when perifusion with the same amino acids was done in the same buffer but in the presence of 1 mM α-MT.

FIGS. 6A to 6C. Effects of SLC6A14 blockade with α-MT in breast cancer cells. In FIG. 6A, MCF10A (a non-malignant mammary epithelial cell line), MCF7 (an ER-positive breast cancer cell line), and MB231 (an ER-negative breast cancer cell line) cells were treated with or without 2.5 mM α-MT for 24, 48, or 72 h and then used for analysis of asparagine synthetase (ASNS) and CHOP mRNA levels. In FIG. 6B, MCF7, MB231 and MCF10A cells were treated with or without 2.5 mM α-MT for 48 h and then used for immunocytochemical analysis of the constitutively expressed LC-3. In FIG. 6C, MCF7 cells were treated with (“b” and “c”) or without (“a”) α-MT (2.5 mM) for 48 h and then used for electron microscopy. Arrows indicate sequestration of cytoplasm and organelles in pre-autophagosomes and autophagosome, and the asterisk indicates condensed chromatin. N, nucleus.

FIGS. 7A and 7B. Induction of autophagy in MCF7 cells by α-MT. FIG. 7A shows autophagosomal proteolysis of ¹⁴C-labeled proteins. The concentrations of α-MT and 3-MA, when present, were 2.5 mM and 10 mM. The treatment time was 48 h. The extent of proteolysis was determined by the radioactivity present in the protein-free supernatant. “a” represents significantly different from proteolysis in the absence of α-MT and 3-MA (p<0.001); “b” represents significantly different from proteolysis in the absence of α-MT and 3-MA (p<0.05); and “c” represents significantly different from proteolysis in the presence of α-MT alone (p<0.01). In FIG. 7B, MCF7 cells were transfected with an GFP-LC3 expression plasmid, and 24 h later, the cells were treated with or without α-MT (2.5 mM) in the presence or absence of 3-MA (10 mM) for 48 h. At the end of the treatment, cells were fixed, and the percent of cells with punctate localization of the ectopically expressed GFP-LC3 was quantified. “a” represents compared to control; “b” represents compared to treatment with α-MT alone; and “c” represents compared to treatment with α-MT alone; *, p<0.001.

FIGS. 8A and 8B. Influence of α-MT on ASNS and CHOP mRNA levels in control MCF7 cells and in MCF7 cells with shRNA-induced silencing of SLC6A14. In FIG. 8A, five different SLC6A14-specific shRNAs were tested in MCF7 cells using a lentivirus-based system to monitor their efficacy to silence SLC6A14. SLC6A14 mRNA levels were measured by RT-PCR with GAPDH as an internal control. c, control. In FIG. 8B, SLC6A14 was silenced in MCF7 cells with shRNA-4 using a lentivirus-based system and then treated without or with α-MT (2.5 mM, 48 h). RNA isolated from the cells was then used for RT-PCR to monitor the levels of asparagine synthetase (ASNS) and CHOP mRNAs.

FIGS. 9A to 9D. Influence of α-MT on mTOR activity In FIG. 9A, MCF7 and MB231 cells were treated without or with α-MT (2.5 mM) for 24 h, 48 h, or 72 h, and then the cell lysates were used for western blot. Influence of α-MT with or without 3-MA on cell death. In FIG. 9B, MCF7, MB231, and MCF10A cells were treated for 48 h without or with α-MT (2.5 mM), 3-MA (10 mM), or α-MT (2.5 mM) plus 3-MA (10 mM), and then analyzed for cell death by propidium iodide (PI) staining and FACS-based cell cycle analysis. In FIG. 9C, MCF7 cells were treated for 48 h without or with α-MT (2.5 mM), 3-MA (10 mM), or α-MT (2.5 mM) plus 3-MA (10 mM), and then analyzed for apoptotic cell death by annexin V labeling. In FIG. 9D, MCF7 and MB231 cells were treated without or with 2.5 mM α-MT for 24 h, 48 h, or 72 h. Cell lysates were then used for western blot analysis of lamin A degradation product using an antibody that recognizes specifically the proteolytic cleavage product. β-Actin was used as an internal control.

FIGS. 10A to 10D. In vivo efficacy of α-MT in the treatment of ER-positive breast cancer. ZR75.1 (FIG. 10A), MCF7 (FIG. 10B), and MB-231 (FIG. 10C) cells were injected subcutaneously (sc) into Balb/c nude mice (10×10⁶ cells per injection site). For MCF7 cells, slow-releasing estrogen pellets were implanted to support the tumor growth. For each cell line, the control group received water with sucrose and the treatment group received α-MT (2 mg/ml) in water with sucrose. Tumor size was measured periodically. NS, not statistically significant (p>0.05); *, p<0.005; two-tailed unpaired Student's t test. In FIG. 10D, Balb/c nude mice were divided into two groups; the control group received water with sucrose and the treatment group received α-MT (2 mg/ml) in water with sucrose for 2 weeks. Mice were then bled and α-MT in plasma was determined by HPLC.

FIG. 11. Estrogen-dependent suppression of the efficacy of α-MT to induce ASNS and CHOP expression in MCF7 cells. MCF7 cells were cultured in a phenol red-free medium in the absence or presence of estradiol (E2, 10 nM) for 24 h and then treated without or with α-MT (2.5 mM, 24 h). The cells were then used for analysis of SLC6A14, asparagine synthetase (ASNS), and CHOP mRNA levels by RT-PCR. Hypoxanthine/guanine phosphoribosyltransferase-1 (HPRT1) mRNA levels were used as an internal control.

### DETAILED DESCRIPTION

The present invention includes methods of treating estrogen receptor positive breast cancer in a subject, the method including administering to the subject a composition including an inhibitor of the ATB^{0,+} amino acid transporter. In some aspects, the estrogen receptor positive breast cancer is metastatic.

The present invention includes methods of killing estrogen receptor positive breast cancer cells, the method including contacting the cells with a composition including an inhibitor of the ATB^{0,+} amino acid transporter. In some aspects, the estrogen receptor positive breast cancer is metastatic.

The present invention includes methods of inducing amino acid deprivation and/or autophagy in a cancer cell, the method including contacting the cancer cell with an inhibitor of the ATB^{0,+} amino acid transporter. In some aspects, the cancer is a breast cancer. In some aspects, the breast cancer is an estrogen receptor positive breast cancer. In some aspects, the cancer is metastatic.

The present invention includes methods of inducing apoptosis in a cancer cell, the method including contacting the cancer cell with an inhibitor of the ATB^{0,+} amino acid transporter. In some aspects, the cancer is a breast cancer. In some aspects, the breast cancer is an estrogen receptor positive breast cancer. In some aspects, the cancer is metastatic.

The present invention includes methods of inducing apoptosis in a cancer cell, the method including contacting the cancer cell with an inhibitor of the ATB^{0,+} amino acid transporter and an inhibitor of autophagy. In some aspects, the cancer is a breast cancer. In some aspects, the breast cancer is an estrogen receptor positive breast cancer. In some aspects, the cancer is metastatic.

In some aspects of the methods of the present invention, the inhibitor of the ATB0,+ amino acid transporter includes a tryptophan derivative.

In some aspects of the methods of the present invention, the inhibitor of the ATB0,+ amino acid transporter includes alpha methyl tryptophan.

In some aspects of the methods of the present invention, the composition includes the L isomer of alpha methyl tryptophan and does not includes the D isomer of alpha methyl tryptophan.

In some aspects of the methods of the present invention, the method further includes administration of an inhibitor of autophagy. In some aspects, an inhibitor of autophagy include 3-methyladenine, suppressive miR-101, lucanthone, chloroquine, hydroxychloroquine, N-acetyl-L-cysteine, L-asparagine, bafilomycin A1 from Streptomyces griseus, catalase, JRF 12 N2,N4-dibenzylquinazoline-2,4-diamine (DbeQ), (2S,3S)-trans-Epoxysuccinyl-L-leucylamido-3-methylbutane ethyl ester EST (E-64d), leupeptin hemisulfate, 2-(4-Morpholinyl)-8-phenyl-1(4H)-benzopyran-4-one hydrochloride (LY-294,002), pepstatin A, thapsigargin, or wortmannin from Penicillium funiculosum.

In some aspects of a method of the present invention, the method further includes administration of an additional therapeutic agent.

In some aspects of the methods of the present invention, the composition is formulated for parenteral delivery. In some aspects, the composition is formulated for enteral delivery.

Inhibitors of the amino acid transporter ATB^{0,+} have been identified (see, for example, U.S. Pat. No. 8,436,039 (“Inhibitors of the ATB(0,+) Transporter and Uses Thereof,” which is herein incorporated by reference). These inhibitors have been shown to be effective in inhibiting the growth and viability of cancer cells. The amino acid transporter ATB^{0,+} (also referred to herein as “ATB^{0,+} transporter,” “the ATB^{0,+} transporter,” “ATB0,+,” “‘ATB 0,+,” “ATB(0,+),” “ATB (0,+),” and “SLC6A14”) is an amino acid transporter with special functional features (Ganaphthy and Ganaphthy, 2005, Curr Drug Targets Immune Endocr Metab Disord; 5:357-364). It has broad substrate selectivity, transporting 18 of the 20 proteinogenic amino acids. The only amino acids that are not substrates for this transporter are glutamate and aspartate, amino acids that are non-essential.

The transport function of ATB^{0,+} is energized by three different driving forces, namely an Na⁺ gradient, a Cl⁻ gradient and membrane potential. Theoretically, this transporter has the ability to concentrate its substrates inside the cells more than 1000-fold. ATB^{0,+} has been cloned from human and rodent tissues, and the function of the cloned transporter has been characterized in heterologous expression systems (Sloan and Mager, 1999, J Biol Chem; 274:23740-23745; Nakanishi et al., 2001, J Physiol; 532:297-304; Uchiyama et al., 2008, J Cell Physiol; 214:645-654). According to the HUGO (Human Genome Organisation) nomenclature, ATB^{0,+} is identified as SLC6A14 (solute carrier family 6 member 14; i.e. the 14th member of the solute carrier gene family SLC6). Functional expression in Xenopus laevis oocytes has demonstrated that the transport process mediated by ATB^{0,+} is electrogenic, associated with the transfer of net positive charge into the oocytes (Sloan and Mager, 1999, J Biol Chem; 274:23740-23745; Nakanishi et al., 2001, J Physiol; 532:297-304).

Recent studies have demonstrated the therapeutic potential of this transporter capitalizing on its ability to transport a variety of pharmacological agents into cells, including NOS (nitric oxide synthase) inhibitors (Hatanaka et al., 2001, J Clin Invest; 107:1035-1043) and amino acid-based prodrugs of the antiviral agents acyclovir and ganciclovir (Hatanaka et al., 2004, J Pharmacol Exp Ther; 308:1135-1147; Umapathy et al., 2004, Pharm. Res. 21, 1303-1310). See also, U.S. patent application Ser. Nos. 10/467,893 and 11/813,343.

There is emerging evidence for tumor-associated up-regulation of ATB^{0,+} (Gupta et al., 2005, Biochim Biophys Acta; 1741:215-223; Gupta et al., 2006, Gynecol Oncol; 100:8-13). The expression of this transporter is markedly induced in colorectal cancer (Gupta et al., 2005, Biochim Biophys Acta; 1741: 215-223) and cervical cancer (Gupta et al., 2006, Gynecol Oncol; 100:8-13), and the up-regulation is demonstrable at the mRNA level as well as at the protein level. The ability of the transporter to recognize structurally diverse pharmacological agents lends credence to its therapeutic potential in cancer treatment. None of the other known amino acid transporters in mammalian cells shares these features.

Tumor cells have a unique metabolic need for amino acids to support rapid growth. This is achieved by facilitation of cell cycle and resistance to apoptosis Enhanced cell proliferation places increased demand for nutrients to serve as the building blocks for the synthesis of macromolecules (DNA, RNA, proteins, and lipids) and as the carbon source for generation of metabolic energy in tumor cells. These nutrients include glucose, amino acids, fatty acids, vitamins, and micronutrients such as trace elements. Most of these nutrients are hydrophilic and do not permeate easily across the plasma membrane in mammalian cells. Uptake of hydrophilic nutrients into cells requires specific transporters in the plasma membrane. Even fatty acids, which are hydrophobic, are taken up into cells via specific transporters. Tumor cells employ various mechanisms to satisfy their increased demand for nutrients. Vascularization in solid tumors enhances the blood flow, thus increasing the availability of blood-borne nutrients to tumor cells.

The entry of nutrients into tumor cells is enhanced by upregulation of specific transporters in the plasma membrane. In some instances, the same signaling events that promote vascularization participate also in the upregulation of nutrient transporters, thus coordinating the availability of nutrients with their entry into tumor cells. Since the ability of tumor cells to support their increased demand for nutrients is obligatory for their growth, the pathways involved in this process have potential as drug targets for the treatment of cancer.

With the present invention, inhibitors of the ATB^{0,+} transporter are used in methods of treating cancer, inflammatory diseases, and other conditions, including, but not limited to, ulcerative colitis and inflammatory bowel disease. With the inhibition of amino acid transport, cells cannot obtain essential nutrients and cannot proliferate fast enough to sustain their growth. Thus, with the present invention it will be possible to starve tumor cells to death by interfering with the availability of essential nutrients and their entry into cells. Such nutrient deprivation presents a new strategy to kill cells, including, but not limited to cancer cells, inflamed cells, and cells of the immune system.

The present invention demonstrates that the expression of the ATB^{0,+} transporter is induced and increased compared to normal cells in a variety of cancers. This observation increases the potential of inhibitors as chemotherapeutic agents. Since tumor cells induce these transporters specifically for their unique metabolic needs, normal cells are expected to be relatively resistant to the therapeutic actions of such compounds, thus reducing undesirable side effects.

As used herein, an inhibitor (also referred to herein as a “blocker”) is an agent capable of preventing or decreasing amino acid transport by the ATB^{0,+} transporter. Inhibition of ATB0,+ may occur at any time during protein production and function. For example, inhibition may occur at the transcriptional, translational, and post-translational level. Means of inhibiting uptake may include, without limitation, blocking the interaction between the amino acid and the transporter by competitive binding for the substrate binding site or by binding to the transporter and providing a physical barrier for the amino acid. In another embodiment, the inhibitor may block amino transport by allowing uptake but preventing, for example, electrical current induction.

An inhibitor may be a competitive, noncompetitive, or irreversible inhibitor. A competitive inhibitor is a compound that reversibly inhibits transport activity at the catalytic site, a noncompetitive inhibitor is a compound that reversibly inhibits transport activity at a non-catalytic site, and an irreversible inhibitor is a compound that irreversibly destroys transport activity by forming a covalent bond with the tranporter.

Inhibitors of the ATB^{0,+} transporter include, but are not limited to, small molecules, antibodies, peptides, nucleic acid molecules (including, for example, an antisense molecule, a PNA, or an RNAi), and peptidomimetics. An inhibitor of the ATB^{0,+} transporter may be identified and characterized using a variety of methods, including, but not limited to any of those described herein.

An inhibitor of the ATB^{0,+} transporter may be a small molecule. In some embodiments of the present invention, an inhibitor of the ATB^{0,+} transporter is an amino acid or a derivative of an amino acid. For example, an inhibitor may be a tryptophan derivative.

In some embodiments, the inhibitor is methyltryptophan or a derivative thereof. In a preferred embodiment the inhibitor is an alpha-methyltryptophan (also referred to herein as alpha-MT, a-MT, a-methyltryptophan, α-methyltryptophan, α-MT, or αMT) or a derivative thereof. Typically, the indole side chain unique to tryptophan is present on the α-carbon (i.e. the chiral carbon) and may be referred to as an α-amino acid. The inhibitor of the present invention may exist as either the D-isomer, the L-isomer, or a combination thereof. Derivations of α-methyltryptophan may occur independently or in combination. Examples of derivations include, without limitation, substitution of the methyl group on the α-carbon with an ethyl, propyl, butyl, pentyl, or longer carbon chain. The carbon chain may be straight or it may be branched. Optionally, the methyl group or the substituted carbon chain may be present on the β-carbon. Another example of a derivation of the inhibitor may include, without limitation, the addition of groups on the aromatic carbons. For example, a methyl or a longer carbon chain may be present on one or more of the C1, C2, C3, C4, C5, C6, or C7 carbon(s) of the indole ring. Alternatively, a hydroxyl (—OH) or a halogen (such as bromine, fluorine, chlorine, or iodine) may be present on one or more of the C1, C2, C3, C4, C5, C6, or C7 carbon(s) of the indole ring.

In some embodiments, an inhibitor of the ATB transporter, including, but not limited to alpha methyltryptophan and derivatives thereof, may be a racemic mixture of an inhibitor, an isolated D isomer of an inhibitor, or an isolated L isomer of an inhibitor. For example, inhibition of the ATB^{0,+} transporter may be accomplished by contacting the transporter with a composition that includes a mixture of the D and L isomers of the inhibitor, including, for example, a racemic mixture Inhibition of the ATB^{0,+} transporter may be accomplished by contacting the transporter with a composition that includes one enantiomer of an inhibitor, but does not include the other enantiomer. For example, inhibition of the ATB^{0,+} transporter may be accomplished by contacting the transporter with a composition that includes the D isomer, but does not include the L isomer of the inhibitor. Such a composition consists essentially of the D isomer of the inhibitor. Inhibition of the ATB^{0,+} transporter may be accomplished by contacting the transporter with a composition that includes the L isomer, but does not include the D isomer of the inhibitor. Such a composition consists essentially of the L isomer. The purification of D and L isomers can be carried out, for example, as described in Example 5 of U.S. Pat. No. 8,436,039.

An inhibitor of the ATB^{0,+} transporter may be a nucleic acid molecule. As used herein a nucleic acid molecule may be, for example, deoxyribonucleotides and ribonucleotides. The nucleotides of the nucleic acid molecule may be naturally occurring, synthetic, or modified nucleotides. A nucleic acid molecule may be modified or tagged.

A nucleic acid molecule may be designed to inhibit ATB^{0,+} mediated amino acid transport function by binding to the SLC6A14 gene in a manner that prevents transcription or prevents production of a functional transcript. For example, the nucleic acid molecule may target the start codon or region upstream of the start codon such that transcription does not occur. Alternatively, the nucleic acid molecule may target any portion of SLC6A14 that results in a transcript fragment which cannot be translated. The process of nucleic acid transcription and the cellular machinery necessary for nucleic acid transcription are well-known in the art. The nucleic acid molecule of the invention may prevent transcription of SLC6A14 at any point in the transcription process.

A nucleic acid molecule may also be designed to inhibit ATB^{0,+} by binding to the SLC6A14 transcript in a manner that prevents translation or prevents production of a functional protein. For example, DNA, RNA, or hybrid molecules (i.e., consisting of both DNA and RNA) may be complementary to any portion of the SLC6A14 transcript including, without limitation, the 3′ untranslated region (UTR), the 5′ UTR, any exon, or any intron. Hybridization of the nucleic acid molecule of the invention to any portion of the SLC6A14 transcript prevents translation or results in the translation of a non-functional protein. The process of protein translation and the cellular machinery necessary for translation are well-known in the art. The nucleic acid molecule of the invention may prevent translation of ATB 0,+ at any point in the translation process.

Nucleic acid molecules may be delivered as either single-stranded or double-stranded molecules. The nucleic acid molecules may be delivered as hybrids. Alternatively, the nucleic acid molecules may be incorporated into an expression vector. Expression vectors are well-known in the art. Typical expression vectors are DNA-based and produce an RNA transcript. However, vectors may also be RNA-based and may produce either an RNA transcript or a cDNA fragment.

An inhibitor of the ATB^{0,+} transporter may be an antibody. Such an antibody may, for example, bind specifically to ATB^{0,+} with a binding affinity that reduces or prevents the transporter function of ATB^{0,+}. Such an antibody may, for example, bind specifically to ATB^{0,+} with a binding affinity that prevents or inhibits the binding of a substrate to the transporter. An antibody of the present invention may bind to ATB^{0,+} transporter and not bind to other transporter polypeptides.

An antibody of the present invention may bind to the C-terminal tail of the transporter and not bind to the N-terminal portion of the transporter. For example, an antibody may bind to the C-terminal half of the transporter and not bind to the N-terminal half of the transporter. An antibody of the present invention may bind to the N-terminal tail of the transporter and not bind to the C-terminal portion of the transporter. For example, an antibody may bind to the N-terminal half of the transporter and not bind to the C-terminal half of the transporter.

As used herein, specific binding means binding that is measurably different from a non-specific interaction. Specific binding can be measured, for example, by determining binding of a molecule compared to binding of a control molecule, which generally is a molecule of similar structure that does not have binding activity, for example, an antibody that binds a distinct epitope or antigen. Specificity of binding also can be determined, for example, by competition with a control molecule, for example, competition with an excess of the same molecule. In this case, specific binding is indicated if the binding of a molecule is competitively inhibited by itself. Thus, specific binding between an antibody and antigen is measurably different from a non-specific interaction and occurs via the antigen binding site of the antibody.

As used herein, selective binding refers to a binding interaction that is both specific and discriminating between molecules, for example, an antibody that binds to a single molecule or closely related molecules. For example, an antibody can exhibit specificity for an antigen that can be both specific and selective for the antigen if the epitope is unique to a molecule. Thus, a molecule having selective binding can differentiate between molecules, as exemplified by an antibody having specificity for an epitope unique to one molecule or closely related molecules.

As used herein the term “binding affinity” is intended to mean the strength of a binding interaction and includes both the actual binding affinity as well as the apparent binding affinity. The actual binding affinity is a ratio of the association rate over the disassociation rate. Therefore, conferring or optimizing binding affinity includes altering either or both of these components to achieve the desired level of binding affinity. The apparent affinity can include, for example, the avidity of the interaction. For example, a bivalent heteromeric variable region binding fragment can exhibit altered or optimized. As used herein, the term “substantially the same” when used in reference to binding affinity is intended to mean similar or identical binding affinities where one molecule has a binding affinity that is similar to another molecule within the experimental variability of the affinity measurement. The experimental variability of the binding affinity measurement is dependent upon the specific assay used and is known to those skilled in the art.

An antibody may be an intact antibody, an antibody binding fragment, or a chimeric antibody. A chimeric antibody may include both human and non-human portions. An antibody may be a polyclonal or a moncoclonal antibody. An antibody may be a derived from a wide variety of species, including, but not limited to mouse and human. An antibody may be a humanized antibody. An antibody may be linked to another functional molecule, for example, another peptide or protein, a toxin, a radioisotype, a cytotoxic agent, cytostatic agent, a polymer, such as, for example, polyethylene glycol, polypropylene glycol or polyoxyalkenes.

The antibodies of the present invention include various antibody fragments, also referred to as antigen binding fragments, which include only a portion of an intact antibody, generally including an antigen binding site of the intact antibody and thus retaining the ability to bind antigen. Examples of antibody fragments include, for example, Fab, Fab′, Fd, Fd′, Fv, dAB, and F(ab′)2 fragments produced by proteolytic digestion and/or reducing disulfide bridges and fragments produced from an Fab expression library. Such antibody fragments can be generated by techniques well known in the art. Antibodies of the present invention can include the variable region(s) alone or in combination with the entirety or a portion of the hinge region, CH1 domain, CH2 domain, CH3 domain and/or Fc domain(s).

Antibodies include, but are not limited to, polyclonal antibodies, affinity-purified polyclonal antibodies, monoclonal antibodies, human antibodies, humanized antibodies, chimeric antibodies, anti-idiotypic antibodies, multispecific antibodies, single chain antibodies, single-chain Fvs (scFv), disulfide-linked Fvs (sdFv), Fab fragments, F(ab′) fragments, F(ab′)2 fragments, Fv fragments, diabodies, linear antibodies fragments produced by a Fab expression library, fragments comprising either a VL or VH domain, intracellularly-made antibodies (i.e., intrabodies), and antigen-binding antibody fragments thereof.

An antibody of the present invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Immunoglobulins can have both heavy and light chains. An array of IgG, IgE, IgM, IgD, IgA, and IgY heavy chains can be paired with a light chain of the kappa or lambda form.

An antibody of the invention can be from any animal origin, including birds and mammals. In some embodiments, the antibodies are human, murine, rat, donkey, sheep, rabbit, goat, guinea pig, camel, horse, or chicken antibodies. As used herein, “human” antibodies include antibodies having the amino acid sequence of a human immunoglobulin and include antibodies isolated from human immunoglobulin libraries or from animals transgenic for one or more human immunoglobulins.

The term “polyclonal antibody” refers to an antibody produced from more than a single clone of plasma cells. In contrast “monoclonal antibody” refers to an antibody produced from a single clone of plasma cells. The preparation of polyclonal antibodies is well known. Polyclonal antibodies may be obtained by immunizing a variety of warm-blooded animals such as horses, cows, goats, sheep, dogs, chickens, rabbits, mice, hamsters, guinea pigs and rats as well as transgenic animals such as transgenic sheep, cows, goats or pigs, with an immunogen. The resulting antibodies may be isolated from other proteins by using an affinity column having an Fc binding moiety, such as protein A, or the like.

Monoclonal antibodies can be obtained by various techniques familiar to those skilled in the art. For example, spleen cells from an animal immunized with a desired antigen are immortalized, commonly by fusion with a myeloma cell. Monoclonal antibodies can be isolated and purified from hybridoma cultures by techniques well known in the art. Other known methods of producing transformed B cell lines that produce monoclonal antibodies may also be used. In some embodiments, the antibody can be recombinantly produced, for example, produced by phage display or by combinatorial methods. Such methods can be used to generate human monoclonal antibodies.

A therapeutically useful antibody may be derived from a “humanized” monoclonal antibody. Humanized monoclonal antibodies are produced by transferring one or more CDRs from the heavy and light variable chains of a mouse (or other species) immunoglobulin into a human variable domain, then substituting human residues into the framework regions of the murine counterparts. The use of antibody components derived from humanized monoclonal antibodies obviates potential problems associated with immunogenicity of murine constant regions. The constant region of a humanized monoclonal antibody of the present invention can be that from human immunoglobulin belonging to any isotype. It may be, for example, the constant region of human IgG.

The present invention includes antibody constructs with all of the CDR regions of an anti-transporter antibody, or a subset thereof. For example, the present invention also includes antibody constructs with one, two, or three of the heavy chain CDRs of an anti-transporter antibody, and/or one, two, or three of the CDR regions of the light chain of an anti-transporter antibody, wherein such an antibody retains an anti-transporter binding specificity.

The present invention includes also antibodies that compete with a monoclonal antibody for binding to a transporter, or compete with a monoclonal antibody in the inhibition of transporter function.

Antibodies of the present invention include chimeric antibodies. A chimeric antibody is one in which different portions are derived from different animal species. For example, chimeric antibodies can be obtained by splicing the genes from a mouse antibody molecule with appropriate antigen specificity together with genes from a human antibody molecule of appropriate biological specificity.

Antibodies of the present invention can be produced by an animal, chemically synthesized, or recombinantly expressed. Antibodies of the present invention can be purified by any method known in the art for purification of an immunoglobulin molecule, for example, by chromatography (e.g., ion exchange, affinity, and sizing column chromatography), centrifugation, differential solubility, or by any other standard technique for the purification of proteins.

Antibodies of the present invention can be assayed for immunospecific binding by the methods described herein and by any suitable method known in the art. The immunoassays that can be used include but are not limited to competitive and non-competitive assay systems using techniques such as BIAcore analysis, FACS (Fluorescence activated cell sorter) analysis, immunofluorescence, immunocytochemistry, Western blots, radio-immunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art.

Also included in the present invention are hybridoma cell lines, transformed B cell lines, and host cells that produce the monoclonal antibodies of the present invention; the progeny or derivatives of these hybridomas, transformed B cell lines, and host cells; and equivalent or similar hybridomas, transformed B cell lines, and host cells.

The present invention further provides an isolated polynucleotide molecule having a nucleotide sequence encoding a monoclonal antibody of the invention. The present invention is further directed to an isolated polynucleotide molecule having a nucleotide sequence that has at least 75%, at least 80%, at least 85%, at least 90%, at least 95%, at least 96%, at least 97%, at least 98%, or at least 99% sequence identity to nucleotide sequence encoding a monoclonal antibody of the invention. The invention also encompasses polynucleotides that hybridize under high stringency to a nucleotide sequence encoding an antibody of the invention, or a complement thereof. As used herein “stringent conditions” refer to the ability of a first polynucleotide molecule to hybridize, and remain bound to, a second, filter-bound polynucleotide molecule in 0.5 M NaHPO4, 7% sodium dodecyl sulfate (SDS), and 1 mM EDTA at 65° C., followed by washing in 0.2×SSC/0.1% SDS at 42° C. (see Ausubel et al. (eds.), Current Protocols in Molecular Biology, Vol. 1, Green Publishing Associates, Inc., and John Wiley & Sons, Inc., N.Y. (1989), at p. 2.10.3). Also included in the present invention are polynucleotides that encode one or more of the CDR regions or the heavy and/or light chains of a monoclonal antibody of the present invention, and polynucleotides having a sequence that has at least 90%, at least 95%, at least 96%, at least 97%, at least 98%, or at least 99% sequence identity to a nucleotide sequence including one or more CDRs. General techniques for cloning and sequencing immunoglobulin variable domains and constant regions are well known.

The monoclonal antibodies of the present invention may be coupled directly or indirectly to a therapeutic moiety or a detectable marker by techniques well known in the art. A detectable marker is an agent detectable, for example, by spectroscopic, photochemical, biochemical, immunochemical, or chemical means. Useful detectable markers include, but are not limited to, fluorescent dyes, chemiluminescent compounds, radioisotopes, electron-dense reagents, enzymes, colored particles, biotin, or dioxigenin A detectable marker often generates a measurable signal, such as radioactivity, fluorescent light, color, or enzyme activity. Antibodies conjugated to agents may be used for diagnostic or therapeutic purposes. Examples of such agents include various enzymes, prosthetic groups, fluorescent materials, luminescent materials, bioluminescent materials, radioactive materials, positron emitting metals using various positron emission tomographies, and nonradioactive paramagnetic metal ions. The agent can be coupled or conjugated either directly to the antibody or indirectly, through an intermediate such as, for example, a linker known in the art, using techniques known in the art. Examples of suitable enzymes include horseradish peroxidase, alkaline phosphatase, beta-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; examples of bioluminescent materials include luciferin, and aequorin; and examples of suitable radioactive material include iodine (¹²¹I, ¹²³I, ¹²⁵I, ¹³¹I), carbon (¹⁴C), sulfur (³⁵S), tritium (³H), indium (¹¹¹In, ¹¹²In, ¹¹³mIn, ¹¹⁵mIn), technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru. Techniques for conjugating such therapeutic moieties to antibodies are well-known. Included in the present invention are compositions of one or more of the antibodies of the present invention.

An inhibitor of the present invention may be coupled directly or indirectly to a detectable marker or a therapeutic moiety by techniques well known in the art. A detectable marker is an agent detectable, for example, by spectroscopic, photochemical, biochemical, immunochemical, or chemical means. Useful agents include, but are not limited to, fluorescent dyes, chemiluminescent compounds, radioisotopes, electron-dense reagents, enzymes, colored particles, biotin, or dioxigenin. A detectable marker often generates a measurable signal, such as radioactivity, fluorescent light, color, or enzyme activity. Antibodies conjugated to detectable agents may be used for diagnostic or therapeutic purposes. Examples of detectable agents include various enzymes, prosthetic groups, fluorescent materials, luminescent materials, bioluminescent materials, radioactive materials, positron emitting metals using various positron emission tomographies, and nonradioactive paramagnetic metal ions. The detectable substance can be coupled or conjugated either directly to the antibody or indirectly, through an intermediate such as, for example, a linker known in the art, using techniques known in the art. Examples of suitable enzymes include horseradish peroxidase, alkaline phosphatase, beta-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; examples of bioluminescent materials include luciferin, and aequorin; and examples of suitable radioactive material include iodine (¹²¹I, ¹²³I, ¹²⁵I, ¹³¹I) carbon (¹⁴C), sulfur (³⁵S), tritium (³H), indium (¹¹¹In, ¹¹²In, ¹¹³mIn, ¹¹⁵mIn), technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe) fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru. Techniques for conjugating such therapeutic moieties to antibodies are well-known. Included in the present invention are compositions of one or more of the antibodies of the present invention.

The present invention includes compositions of the inhibitors. A composition may also include, for example, buffering agents to help to maintain the pH in an acceptable range or preservatives to retard microbial growth. Such compositions may also include a pharmaceutically acceptable carrier. As used herein, the term “pharmaceutically acceptable carrier” refers to one or more compatible solid or liquid filler, diluents or encapsulating substances which are suitable for administration to a human or other vertebrate animal. The compositions of the present invention are formulated in pharmaceutical preparations in a variety of forms adapted to the chosen route of administration

The present invention also includes pharmaceutically acceptable salts of inhibitors. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods.

The methods of the present invention may be administered to a patient for the treatment of cancer, including an estrogen-receptor positive cancer. Such a cancer may be a primary cancer or a metastatic cancer. The methods of the present invention may include a step of determining the level or amount of the estrogen receptor expressed on cells in a sample and/or the comparison to expression levels of the transporter on noimal or control cells. The methods of the present invention may include deciding to clinically treat a subject with the administration of an inhibitor the ATB^{0,+} transporter if the cells in the sample express an increased level of the estrogen receptor.

The efficacy of treatment of a cancer may be assessed by any of various parameters well known in the art. This includes, but is not limited to, determinations of a reduction in tumor size, determinations of the inhibition of the growth, spread, invasiveness, vascularization, angiogenesis, and/or metastasis of a tumor, determinations of the inhibition of the growth, spread, invasiveness and/or vascularization of any metastatic lesions, determinations of tumor infiltrations by immune system cells, and/or determinations of an increased delayed type hypersensitivity reaction to tumor antigen. The efficacy of treatment may also be assessed by the determination of a delay in relapse or a delay in tumor progression in the subject or by a determination of survival rate of the subject, for example, an increased survival rate at one or five years post treatment. As used herein, a relapse is the return of a tumor or neoplasm after its apparent cessation, for example, such as the return of leukemia.

Cancers to be treated by the methods of the present invention include cancers that express the ATB^{0,+} transporter. Such expression may be at a level that is increased or enhanced when compared to the level expressed on normal, non-cancerous cells. The methods of the present invention may include a step of determining the level or amount of the ATB^{0,+} transporters expressed on cells in a sample and the comparison to expression levels of the transporter on normal or control cells. The methods of the present invention may include deciding to clinically treat a subject with the administration of an inhibitor the ATB^{0,+} transporter if the cells in the sample express an increased level of the ATB^{0,+} transporter.

As used herein “treating” or “treatment” may include therapeutic and prophylactic treatments. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. The findings of the present invention can be used in methods that include, but are not limited to, methods for treating cancer, methods to treat an infections, methods to increase an immune responses, methods to reduce immunosuppression mediated by regulatory T cells, and methods to increase or stimulate T cell mediated immune responses.

In some embodiments of the methods of the present invention, more than one inhibitors of the ATB^{0,+} transporter may be administered. The compositions of the present invention include a mixture or cocktail of two, three, four, five, or more inhibitors.

With the methods of the present invention, one or more additional therapeutic agents may be administered, in addition to the administration of an inhibitor of the ATB^{0,+} transporter. An inhibitor of the ATB^{0,+} transporter may be administered before, after, and/or coincident to the administration of one or more additional therapeutic agents. An inhibitor of the ATB^{0,+} transporter and one or more additional therapeutic agents may be administered separately or as a part of a mixture or cocktail.

As used herein, an additional therapeutic agent is not an inhibitor of the ATB^{0,+} transporter. As used herein, an additional therapeutic agent is an agent whose use for the treatment of cancer, an infection, or an immune disorder is known the skilled artisan. Additional therapeutic treatments include, but are not limited to, surgical resection, radiation therapy, hormone therapy, vaccines, antibody based therapies, whole body irradiation, bone marrow transplantation, peripheral blood stem cell transplantation, the administration of chemotherapeutic agents (also referred to herein as “antineoplastic chemotherapy agent,” “antineoplastic agents,” or “antineoplastic chemotherapeutic agents”), cytokines, antiviral agents, immune enhancers, tyrosine kinase inhibitors, signal transduction inhibitors, antibiotic, antimicrobial agents, a TLR agonists, such as for example, bacterial lipopolysaccharides (LPS), one or more CpG oligonucleotides (ODN), metabolic breakdown products of tryptophan, inhibitors of a GCN2 kinase, and adjuvants.

In some aspects of the present invention, the administration of one or more inhibitors of the ATB^{0,+} transporter may allow for the effectiveness of a lower dosage of other therapeutic modalities when compared to the administration of the other therapeutic modalities alone, providing relief from the toxicity observed with the administration of higher doses of the other modalities.

The agents of the present invention can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical, or injection into or around the tumor.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal, and intratumoral administration. In this connection, sterile aqueous media that can be employed will be known to those of skill in the art. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA. Such preparation may be pyrogen-free.

For enteral administration, the inhibitor may be administered in a tablet or capsule, which may be enteric coated, or in a formulation for controlled or sustained release. Many suitable formulations are known, including polymeric or protein microparticles encapsulating drug to be released, ointments, gels, or solutions which can be used topically or locally to administer drug, and even patches, which provide controlled release over a prolonged period of time. These can also take the form of implants. Such an implant may be implanted within the tumor.

Therapeutically effective concentrations and amounts may be determined for each application herein empirically by testing the compounds in known in vitro and in vivo systems, such as those described herein, dosages for humans or other animals may then be extrapolated therefrom.

It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

An agent of the present invention may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

With the methods of the present invention, the efficacy of the administration of one or more agents may be assessed by any of a variety of parameters well known in the art.

As used herein, the term “subject” includes, but is not limited to, humans and non-human vertebrates. In preferred embodiments, a subject is a mammal, particularly a human. A subject may be an individual. A subject may be a patient. Non-human vertebrates include livestock animals, companion animals, and laboratory animals Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits.

The methods of the present invention include in vivo and in vitro methods. As used herein “in vitro” is in cell culture and “in vivo” is within the body of a subject.

As used herein, “isolated” refers to material that has been either removed from its natural environment (e.g., the natural environment if it is naturally occurring), produced using recombinant techniques, or chemically or enzymatically synthesized, and thus is altered “by the hand of man” from its natural state.

In some therapeutic embodiments, an “effective amount” of an agent is an amount that results in a reduction of at least one pathological parameter. Thus, for example, in some aspects of the present invention, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

For any method disclosed herein that includes discrete steps, the steps may be conducted in any feasible order. And, as appropriate, any combination of two or more steps may be conducted simultaneously.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### SLC6A14 (ATB0,+) as a Novel and Effective Drug Target for Treatment of Estrogen Receptor-Positive Breast Cancer

SLC6A14, also known as ATB^{0,+}, is an amino acid transporter with unique characteristics. It transports 18 of the 20 proteinogenic amino acids. However, this transporter is expressed only at low levels in normal tissues. The present example shows that the transporter is upregulated specifically in estrogen receptor (ER)-positive breast cancer, demonstrable with primary human breast cancer tissues and human breast cancer cell lines. SLC6A14 is an estrogen/ER target. The transport features of SLC6A14 include concentrative transport of leucine (an activator of mTOR), glutamine (essential for nucleotide biosynthesis and substrate for glutaminolysis), and arginine (an essential amino acid for tumor cells), suggesting that ER-positive breast cancer cells upregulate SLC6A14 to meet their increased demand for these amino acids. Consequently, treatment of ER-positive breast cancer cells in vitro with α-methyl-DL-tryptophan (α-MT), a selective blocker of SLC6A14, induces amino acid deprivation, inhibits mTOR, and activates autophagy. Prolongation of the treatment with α-MT causes apoptosis. Addition of an autophagy inhibitor (3-methyladenine) during α-MT treatment also induces apoptosis. These effects of α-MT are specific to ER-positive breast cancer cells, which express the transporter. The ability of α-MT to cause amino acid deprivation is significantly attenuated in MCF7 cells, an ER-positive breast cancer cell line, when SLC6A14 is silenced with shRNA. In mouse xenograft studies, α-MT by itself is able to reduce the growth of the ER-positive ZR75.1 breast cancer cells. These studies identify SLC6A14 as a novel and effective drug target for the treatment of ER-positive breast cancer.

ATB^{0,+} (Amino acid Transporter responsible for the activity of system B^{0,+}) was named “B^{0,+}” to indicate its broad substrate selectivity (denoted by ‘B’), accepting neutral (denoted by ‘0’ in the superscript) and cationic (denoted by ‘+’ in the superscript) amino acids as substrates (Ganaphthy, V., Inoue, K., Prasad, P. D., and Ganaphthy, M. E. (2004) In: Metabolic and Therapeutic Aspects of Amino Acids in Clinical Nutrition (ed., Cynober, L. A.), 2nd edition, pp. 63-78. CRC Press, Boca Raton, Fla.; and Ganaphthy and Ganaphthy, 2005, Curr Drug Targets Immune Endocr Metab Disord; 5:357-364 1, 2). Its transport function is coupled to a Na⁺ gradient, a gradient, and membrane potential. ATB^{0,+} is identified as SLC6A14 according to the Human Genome Organization nomenclature. This transporter has potential for delivery of a wide variety of drugs and prodrugs into cells (Nakanishi et al., 2001, J Physiol; 532:297-304; Hatanaka et al., 2001, J Clin Invest; 107:1035-1043; Hatanaka et al., 2002, Biochem Biophys Res Commun; 291:291-295; Hatanaka et al., 2004, J Pharmacol Exp Ther; 308:1135-1147; and Umapathy et al., 2004, Pharm Res: 21:1303-1310).

The substrate selectivity of SLC6A14 is interesting. It transports all essential amino acids. The only excluded amino acids are glutamate and aspartate, which are nonessential. It also transports glutamine (an important precursor for nucleotide synthesis) and arginine (an amino acid essential for tumor growth). However, the transporter is expressed only at low levels in normal tissues. Tumor cells have an increased requirement for essential amino acids as well as glutamine and arginine to support their rapid growth. Essential amino acids are obligatory for protein synthesis. Leucine, an essential amino acid, is also a potent activator of mTOR (Wang and Proud, 2009, Trends Cell Biol; 19:260-267). Certain tumor cells metabolize glutamine at a rate far exceeding the requirement for protein and nucleotide synthesis, a phenomenon known as “glutamine addiction” (DeBerardinis et al., 2008, Cell Metab; 7:11-20; Tong et al. 2009, Curr Opin Genet Dev; 19:32-37; and Ganaphthy et al., 2009, Pharmacol Ther; 121:29-40). Glutamine metabolism through a set of biochemical reactions called “glutaminolysis” provides a carbon source for tumor cells, thereby sparing glucose-derived carbon for the synthesis of lipids and other essential biomolecules. Arginine is essential for several types of cancer due to lack of the arginine-synthesizing enzyme argininosuccinate synthetase (Feun et al., 2008, Curr Pharm Des; 14:1049-1057; and Delage et al., 2010, Int J Cancer; 126:2762-2772). Based on these findings, it was hypothesized that the expression of SLC6A14 may be upregulated in cancer to meet their increasing demand for all essential amino acids as well as glutamine and arginine. In support of this hypothesis, past studies have shown that SLC6A14 is upregulated in colon cancer (Gupta et al., 2005, Biochim Biophys Acta; 1741:215-223) and cervical cancer (Gupta et al., 2006, Gynecol Oncol; 100:8-13). The transporter is also upregulated in breast cancer cell lines (Karunakaran et al., 2008, Biochem J; 414:343-355). In the present example, the expression of SLC6A14 in primary breast cancer tissues, its relevance to ER status, and its potential as a drug target for in vivo breast cancer therapy were investigated.

### Experimental Procedures

Materials. L-[¹⁴C]Valine was purchased from Moravek Biochemicals (Brea, Calif.). Polyclonal antibody specific for human SLC6A14 has been described previously (Hatanaka et al., 2002, Biochem Biophys Res Commun; 291:291-295). All other antibodies were purchased from commercial sources. Primary tumor and normal tissues, used for RNA preparation, were obtained from the Georgia Health Sciences University Tumor Bank.

Animals Female athymic Balb/c mice were obtained from Taconic, (Germantown, N.Y.). Frogs were obtained from Xenopus (Ann Arbor, Mich.). Use of animals in these studies adhered to the Guide for the Care and Use of Laboratory Animals (NIH Pub. No. 85-23, revised 1985) and was approved by the Institutional Committee for Animal Use in Research and Education of the Georgia Health Sciences University.

Construction of luciferase reporter plasmid with SLC6A14 promoter. Human genomic DNA was used for PCR to clone the SLC6A14 promoter using the primers 5′-GTCGACCCTGCCTTCAAAGAACTGGTA-3′ (SEQ ID NO:1) and 5′-AAGCTTGCACTCTCCCCTGTTCCTA-3′ (SEQ ID NO:2). Bold/italicized sequences represent SalI and HindIII restriction sites for subcloning the PCR product into the luciferase reporter plasmid pGL3.

Immunohistochemistry. Primary breast tissue sections, purchased from US Biomax, Inc. (Rockville, Md.), were subjected to immunohistochemistry to assess qualitatively the expression levels of SLC6A14 protein in normal and tumor tissues.

RT-PCR. Total RNA was isolated using TRIZOL™ (Invitrogen). 2 μg of RNA was reverse transcribed using the GeneAmp PCR System (Applied Biosystems, Branchburg, N.J.). PCR reaction was performed under optimal conditions specific for each of the primer pairs. GAPDH (glyceraldehyde-3-phosphate dehydrogenase) or HPRT1 (hypoxanthine/guanine phosphoribosyl transferase-1) were used as internal controls. PCR products were size-fractionated on agarose gel, stained with ethidium bromide and imaged using Alpha Imager (Santa Clara, Calif.). Densitometry was performed on PCR bands and normalized to the internal control. Normalized values were used to compare mRNA levels of target genes. Primers used for PCR were as follows. ASNS (asparagine synthetase):

| | |
|---|---|
| 5′-GCACGCCCTCTATGACAATG-3′ | (SEQ ID NO: 3) |
| (upstream) | |
| and | |
| 5′-CTCACTCTCCTCGGCTTT-3′; | (SEQ ID NO: 4) |

CHOP (C/EBP homologous protein, where C/EBP is CCAAT/enhancer binding protein):

| | |
|---|---|
| 5′-GAGAACCAGGAAACGGAAAC-3′ | (SEQ ID NO: 5) |
| (upstream) | |
| and | |
| 5′-GCAGATTCACCATTCGGTC-3′ | (SEQ ID NO: 6) |
| (downstream); | |
| SLC6A14: | |
| 5′-GAAGGAGAAAGTGTCGGCTTCA-3′ | (SEQ ID NO: 7) |
| (upstream) | |
| and | |
| 5′-TACCACCTTGCCAGACGATTTG-3′ | (SEQ ID NO: 8) |
| (downstream); | |
| ER: | |
| 5′-GGCTCCGCAAATGCTACGA-3′ | (SEQ ID NO: 9) |
| (upstream) | |
| and | |
| 5′-CGCCAGACGAGACCAATCATC-3′ | (SEQ ID NO: 10) |
| (downstream); | |
| GAPDH: | |
| 5′-TGGACCTGACCTGCCGTCTA-3′ | (SEQ ID NO: 11 |
| (upstream) | |
| and | |
| 5′-AGGAGTGGGTGTCGCTGTTG-3′ | (SEQ ID NO: 12) |
| (downstream); | |
| HPRT1: | |
| 5′-GCGTCGTTAGCGATGATGAAC-3′ | (SEQ ID NO: 13 |
| (upstream) | |
| and | |
| 5′-CCTCCCATCTCCTTCATGACATCT-3′ | (SEQ ID NO: 14) |
| (downstream). | |

Analysis of SLC6A14 as an estrogen/ER target. The influence of estrogen on SLC6A14 expression was assessed in ER-positive breast cancer cells (ZR75.1 and BT474) by culturing them for several passages in the absence of phenol red, and then monitoring the levels of SLC6A14 mRNA. The effect of estradiol/ER was investigated directly in BT474 cells. These experiments were complemented with a SLC6A14-promoter activity assay in BT474 cells using luciferase as the reporter. Cells were cotransfected with the luciferase reporter plasmid containing the SLC6A14 promoter upstream of the luciferase cDNA and a β-galactosidase expression plasmid (ratio of the two plasmid DNAs, 4:1). Luciferase activity was measured in cell lysates using Luciferase Assay Reagent (Promega). β-galactosidase activity in the same cell lysates was used for normalization of the promoter activity for differences in transfection efficiency.

Analysis of SLC6A14 transport function in X. laevis oocytes. SLC6A14-mediated transport of leucine, glutamine, and arginine and its blockade by α-MT were studied using the X. laevis oocyte heterologous expression system as described previously (Hatanaka et al., 2001, J Clin Invest; 107:1035-1043; and Karunakaran et al., 2008, Biochem J; 414:343-355). Human cloned SLC6A14 cDNA was used in these studies.

Analysis of autophagy. Autophagy was analyzed as described previously (Periyasamy-Thandavan et al., 2010, Autophagy; 6:19-35; and Samaddar et al., 2008, Mol Cancer Ther; 7:2977-2987) by monitoring the localization pattern of constitutively expressed LC3 as well as that of ectopically expressed GFP-LC3. In the case of the constitutively expressed LC3, cells were treated without or with α-MT, and then used for immunocytochemical analysis of LC3 localization pattern. For ectopically expressed LC3, cells were transfected with a GFP-LC3 expression plasmid. Briefly, cells were plated on a coverslip to reach ˜50% confluence and transfection of these cells was done with 1 μg of the plasmid using FuGENE transfection reagent (Roche, Nutley, N.J.). The cells were then maintained in culture for 24 h to reach 80-90% confluence prior to treatment with α-MT in the presence or absence of 3-methyladenine (3-MA). At the end of the treatment, cells were fixed with 4% paraformaldehyde and examined by fluorescence microscopy to count the number of cells with punctate GFP-LC3. Autophagosomal proteolysis was quantified as described previously (Periyasamy-Thandavan et al., 2010, Autophagy; 6:19-35). For electron microscopy, control and treated MCF7 cells were fixed with a solution consisting of 2.5% glutaraldehyde, 1% formaldehyde and 0.1 M cacodylate buffer, pH 7.4. The samples were then post-fixed in 1% osmium tetroxide/0.1 M cacodylate buffer and embedded in epoxy resin Epon. Samples were then cut into 0.1-μm sections, stained with uranyl acetate/lead citrate, and examined with a transmission electron microscope.

Apoptosis assay. Fluorescence-activated cell sorting (FACS) was used to monitor cell death. MCF7 cells were treated with or without α-MT, 3-MA, or α-MT plus 3-MA. Control and treated cells were then fixed in 50% ethanol, treated with 0.1% sodium citrate, 1 mg/ml RNase A, and 50 μg/ml propidium iodide, and then subjected to FACS analysis (FACS Caliber, Becton Dickinson). FITC-labeled annexin V was used to differentiate apoptotic cell death from necrotic cell death.

Migration and invasion assays. The influence of α-MT on cell migration and invasion was determined with ZR75.1 cells. The migration of these cells toward fetal bovine serum was measured using the QCM™ cell migration assay kit (Millipore) according to manufacturer's instructions. Serum-starved ZR75.1 cells were allowed to migrate for 48 h in the presence/absence of α-MT (2.5 mM). After removing the cells on the top of the membrane filter, the inserts were stained with 0.1% crystal violet, washed and dried. The crystal violet dye retained on the filters, representing the cells that migrated to the underside of the membrane filter, was extracted with acetic acid. The absorbance of the extract was measured at 570 nm. Cell invasion assay was performed similarly using the QCM™ cell invasion assay kit (Millipore). The inserts used in this assay were coated with ECMATRIX™, representing extracellular matrix.

shRNA-mediated silencing of SLC6A14. A lentivirus-based system was used for shRNA-mediated silencing of SLC6A14 in MCF7 cells. A set of lentivirus-based shRNAs (five different shRNAs) was purchased from Open Biosystems (RHS4533-NM_{—}007231). Recombinant lentivirus was produced in 293FT cells by cotransfection of empty vector or shRNA plasmid along with the helper vectors, pLP-1, pLP-2 and pVSVG (Invitrogen).

Lipofectamine-2000 was used as the transfection reagent. Lentiviral supernatant was harvested 72 h after transfection and filtered through a 0.45-μm membrane. MCF7 cells were infected for 24 h with lentivirus in a medium containing 8 μg/mlpolybrene and cultured for an additional 24 h. The expression of SLC6A14 was monitored by RT-PCR.

Xenograft in nude mice. Human breast cancer cells (ZR75.1, MCF7, and MB231) were injected s.c. close to the lower mammary gland on the right side of Balb/c nude mice (10×10⁶ cells per injection site). Only for MCF7 cells, slow-releasing estrogen pellets were implanted to support the tumor growth. Implantation of estrogen pellets was done as follows. Seven days prior to injection of tumor cells, mice were anesthetized and 3-mm pellets containing estradiol (0.18 mg/21-day release; Innovative Research of America, Sarasota, Fla.) were implanted s.c. in the animal's back. The pellets provided a continuous release of estradiol to maintain serum concentrations of 150-250 pM, which is in the range of physiological levels seen in mice during the estrous cycle. Mice were then divided into two groups (control and treatment) with 6 mice in each group. The control group received water with sucrose and the treatment group received α-MT (2 mg/ml) in water with sucrose 48 h prior to injection of tumor cells. The treatment group continued to receive α-MT (2 mg/ml) in water throughout the experimental period. Tumor size in control and treatment groups was measured periodically by caliper and area of tumor was calculated using the formula (width²×length)/2.

Measurement of α-MT in mouse plasma. Balb/c nude mice were divided into two groups; the control group received water with sucrose and the treatment group received α-MT (2 mg/ml) in water with sucrose for 2 weeks. Mice were then bled and α-MT in plasma was determined by HPLC as described by Laich et al (Laich et al., 2002, Clin Chem; 48:579-581).

### Results and Discussion

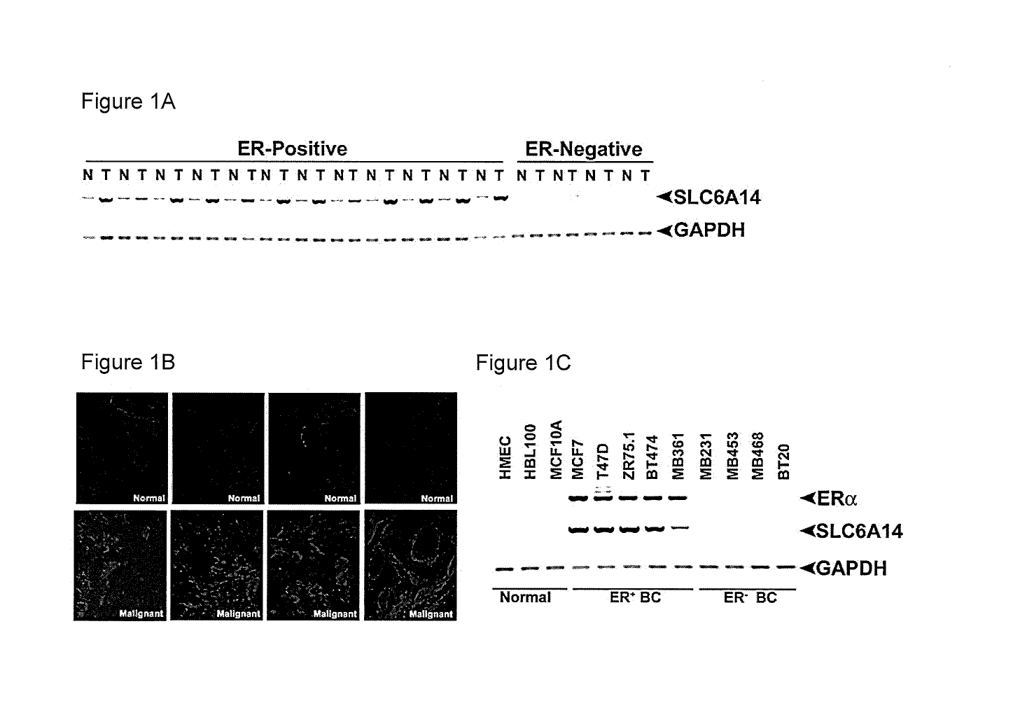

Upregulation of SLC6A14 in ER-positive breast cancer. This example examined the levels of SLC6A14 mRNA in primary breast cancer tissues (12 ER-positive and 4 ER-negative) and adjacent normal breast tissues (FIG. 1A). In each of the ER-positive breast cancer tissue specimens, SLC6A14 was upregulated compared to corresponding normal tissues (9.8±1.0-fold). In contrast, SLC6A14 mRNA was not detectable in ER-negative breast cancer and in corresponding normal tissues. The absence of expression of SLC6A14 in normal tissues adjacent to ER-negative tumors was not surprising because ER was absent not only in these tumor tissues but also in corresponding adjacent normal tissues. The SLC6A14 protein levels were also higher in ER-positive breast cancer than in normal breast tissue (FIG. 1B). The specific upregulation of SLC6A14 in ER-positive breast cancer was reproducible in human breast cancer cell lines (FIG. 1C). The non-malignant mammary epithelial cell lines HMEC, HBL100, and MCF10A as well as the ER-negative breast cancer cell lines MB231, MB453, MB468, and BT20 were negative for ER and SLC6A14 expression. In contrast, the ER-positive breast cancer cell lines MCF7, T47D, ZR75.1, BT474, and MB361 expressed the transporter.

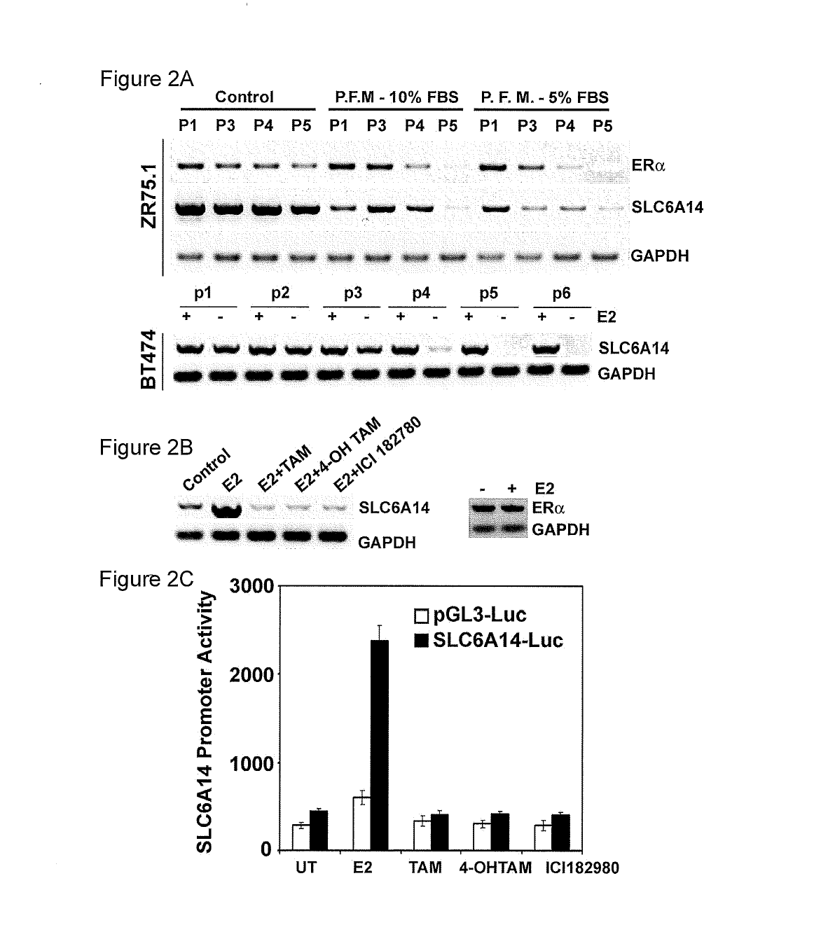

SLC6A14 as an estrogen/ER target. To determine if SLC6A14 is an ER target, the SLC6A14 promoter sequence (˜3 kb) was analyzed. Several putative ER-binding sites were found in the promoter. Then, two ER-positive breast cancer cells (ZR75.1 and BT474) were cultured for 5-6 passages in a culture medium without phenol red and monitored the levels of SLC6A14 mRNA (FIG. 2A). Phenol red is an estrogen mimetic; therefore, if ER induces SLC6A14 expression, absence of phenol red during the culture of ER-positive breast cancer cells is expected to reduce SLC6A14 expression. This was indeed the case. The absence of phenol red in the culture medium led to the downregulation of ER in both cell lines with a corresponding decrease in SLC6A14 expression. Treatment with estradiol increased SLC6A14 mRNA levels in BT474 cells and this effect was abolished completely in the presence of antiestrogens (FIG. 2B). Then, the activity of SLC6A14-promoter (˜3 kb) in BT474 cells was monitored using luciferase as the reporter. Cells were cotransfected with the reporter plasmid and a β-galactosidase expression plasmid, and β-galactosidase activity was used to control for differences in transfection efficiency. Untreated cells showed significant promoter activity, and this activity was enhanced 6-fold by estrogen (FIG. 2C). Antiestrogens blocked this effect. The stimulatory effect of estrogen on luciferase activity was not seen in cells transfected with vector alone. It has been shown by other investigators that chronic treatment (96 h) with estrogen downregulates ER in ER-positive breast cancer cell lines (Kousidou et al., 2008, Mol Oncol; 2:223-232). In the present study, treatment of BT474 cells with estradiol for only 24 h did not have any noticeable effect on the expression of ER (FIG. 2B).

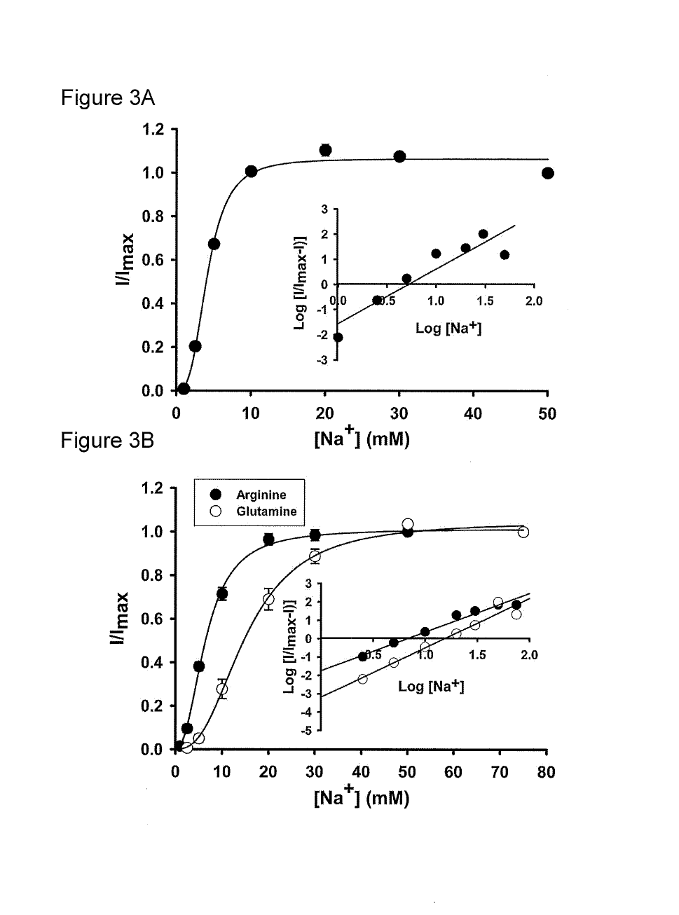

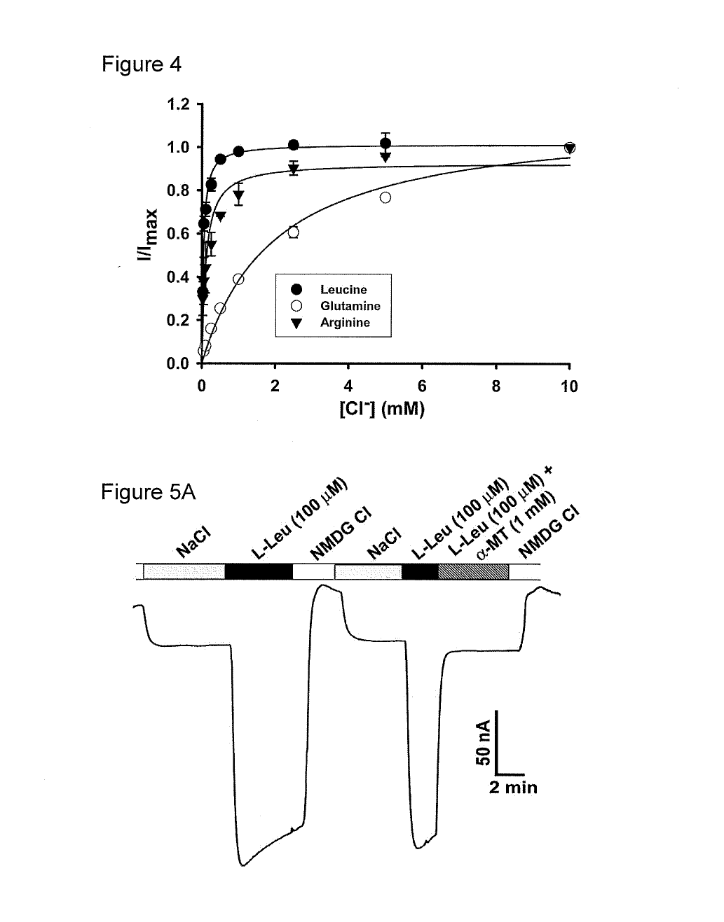

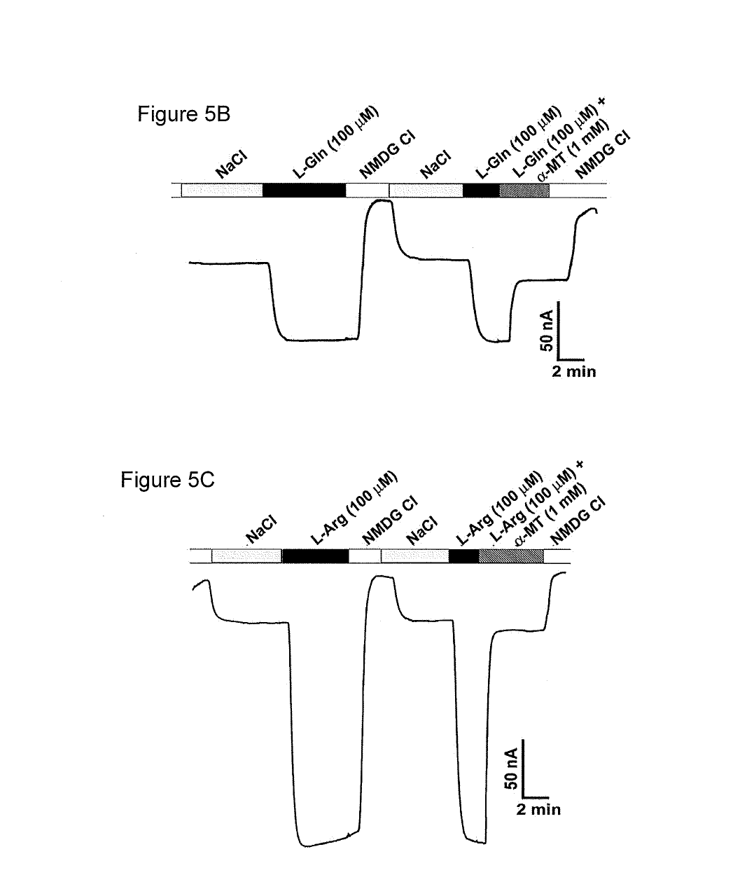

Transport of leucine, glutamine, and arginine via SLC6A14 and its blockade by α-MT. While the characteristics of leucine transport via SLC6A14 are known with regard to Na⁺- and Cl⁻-dependence and Na⁺:Cl⁻: amino acid stoichiometry (Sloan and Mager, 1999, J Biol Chem; 274:23740-23745), such information is not available for glutamine and arginine. Because of the charge differences between leucine/glutamine and arginine, the Nat and Cl⁻-dependence and Na⁺:Cl⁻: amino acid stoichiometry may or may not be the same for these three amino acids. Therefore, SLC6A14-mediated transport of leucine, glutamine, and arginine was investigates electrophysiologically using the oocyte expression system. Perifusion of SLC6A14-expressing oocytes with all three amino acids induced inward currents in the presence of NaCl; such currents were not detectable in the absence of either Na⁺ or Cl⁻, indicating that both ions are obligatory for transport. With each of these three amino acids, the Na⁺-activation kinetics exhibited a sigmoidal relationship (FIG. 3), suggesting involvement of more than one Na⁺ in the activation process. The value for the Hill coefficient (i.e., the number of Na⁺ involved in the activation process) was two for all three amino acids (2.2 0.4 for leucine, 2.4 0.3 for glutamine, and 2.1 0.1 for arginine) In contrast, the Cl⁻-activation kinetics was hyperbolic (i.e., Hill coefficient=1) for all three amino acids, suggesting involvement of one Cl⁻ in the activation process (FIG. 4). Thus, the Na⁺:Cl⁻:amino acid stoichiometry was 2:1:1 irrespective of the net charge on the amino acid substrate. Then, the effects of α-MT on SLC6A14-mediated transport of leucine, glutamine, and arginine was investigated. Perifusion of SLC6A14-expressing oocytes with 100 M leucine, glutamine, or arginine induced inward currents, and these currents were blocked by 1 mM α-MT (FIG. 5).

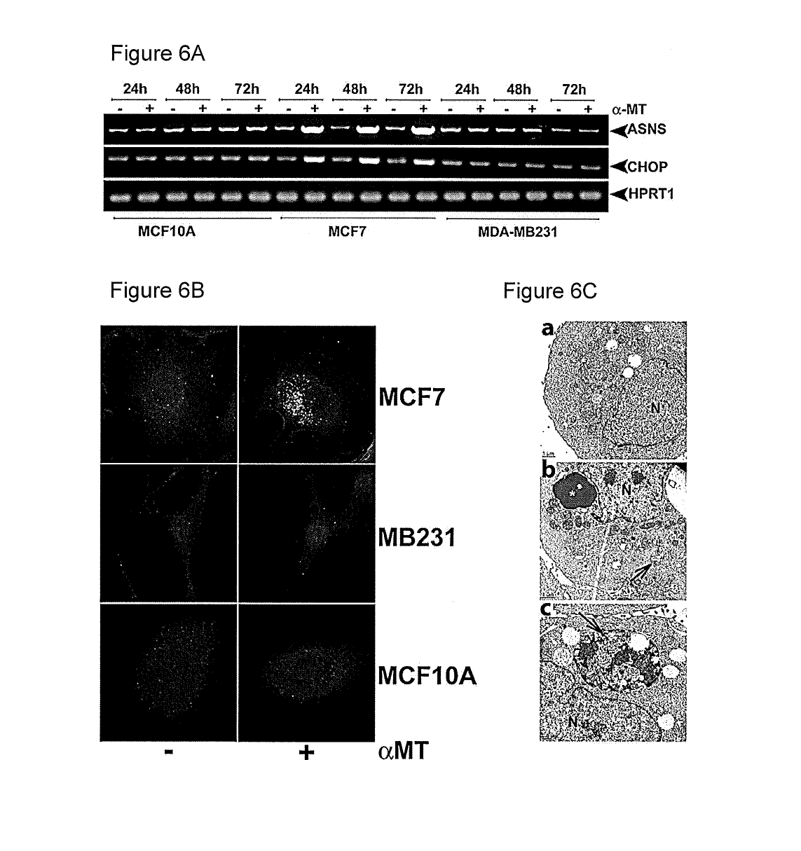

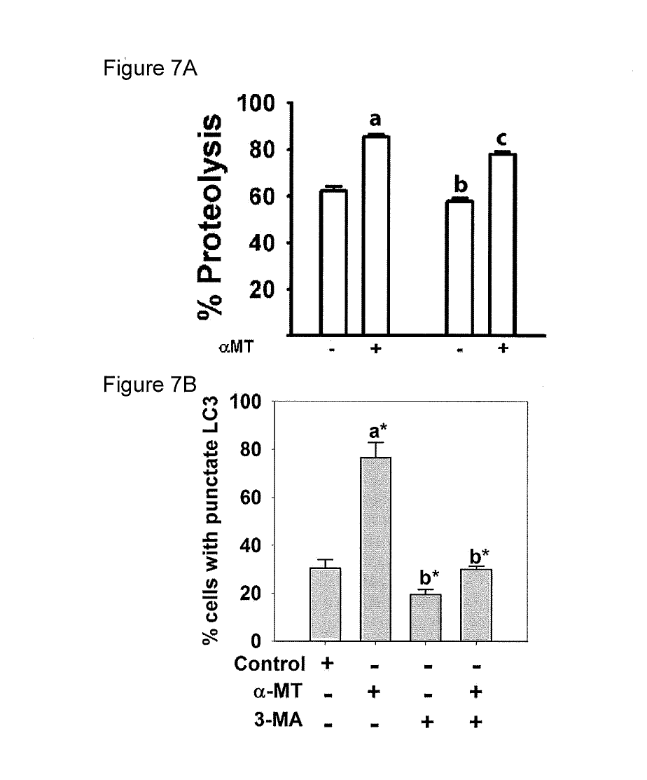

Consequences of blockade of SLC6A14 in breast cancer cells. To test whether blockade of SLC6A14 causes amino acid deprivation in SLC6A14-positive breast cancer cells, MCF7 cells were treated with α-MT (2.5 mM) for 24, 48, or 72 h and the levels of asparagine synthetase mRNA and CHOP mRNA monitored as a readout for amino acid deprivation (Kilberg et al., 2009, Trends Endocrinol Metab; 20:436-443). It was found that α-MT treatment increased the levels of these mRNAs in MCF7 cells but not in SLC6A14-negative MB231 (a cancer cell line) and MCF10A (a non-malignant cell line) cells (FIG. 6A). Treatment of MCF7 cells with α-MT induced autophagy as evident from the punctate localization of the constitutively expressed LC3 in immunocytochemical analysis (FIG. 6B) and the presence of autophagosomes in electron microscopic analysis (FIG. 6C). Under identical conditions, MB231 and MCF 10A cells did not undergo autophagy. The induction of autophagosomal proteolysis in MCF7 cells by α-MT was further substantiated by increased proteolysis of 14C-labeled proteins (FIG. 7A). This α-MT-induced proteolysis was inhibited significantly by cotreatment with 3-MA, a blocker of autophagy (FIG. 7A). The inhibition of autophagy by 3-MA was more clearly evident when the punctate localization of ectopically expressed GFP-LC3 was taken as the marker of autophagy (FIG. 7B). Here MCF7 cells, transiently transfected with GFP-LC3, were treated for 48 h with α-MT (2.5 mM), 3-MA (10 mM), or α-MT (2.5 mM) plus 3-MA (10 mM), and then the percent of cells showing punctate localization of LC3 was determined by fluorescence microscopy. 3-MA was able to block >90% of autophagy induced by α-MT.

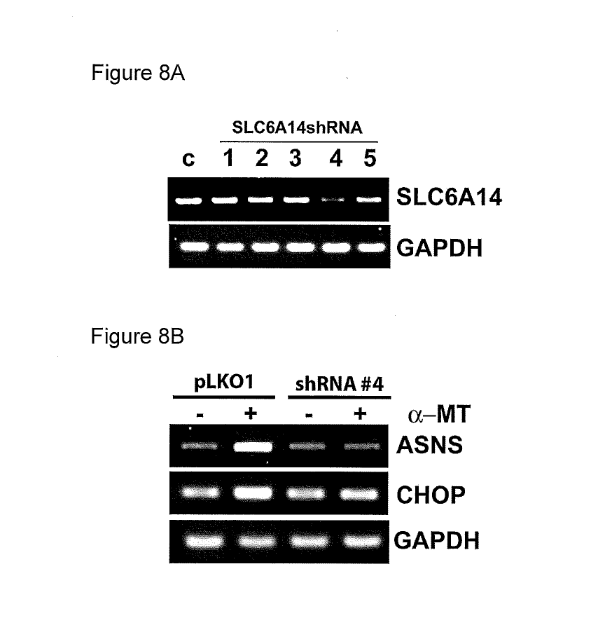

To determine whether the induction of amino acid deprivation by α-MT in ER-positive breast cancer cells was due to blockade of SLC6A14, the expression of the transporter was silenced in MCF7 cells, an ER-positive breast cancer cell line, using shRNA. Among the five SLC6A14-selective shRNAs tested, only shRNA #4 was able to silence the transporter expression markedly (FIG. 8A). Then, MCF7 cells were transfected with either empty vector or the vector carrying the shRNA #4 and then treated the cells without or with α-MT (2.5 mM, 48 h) to monitor the expression levels of asparagine synthetase and CHOP as a readout for amino acid deprivation. In control cells transfected with empty vector, the expression levels of asparagine synthetase and CHOP were increased by α-MT treatment (FIG. 8B). This effect was markedly attenuated in cells transfected with the vector carrying the shRNA #4. These results show that the amino acid deprivation induced by α-MT occurs via blockade of SLC6A14.

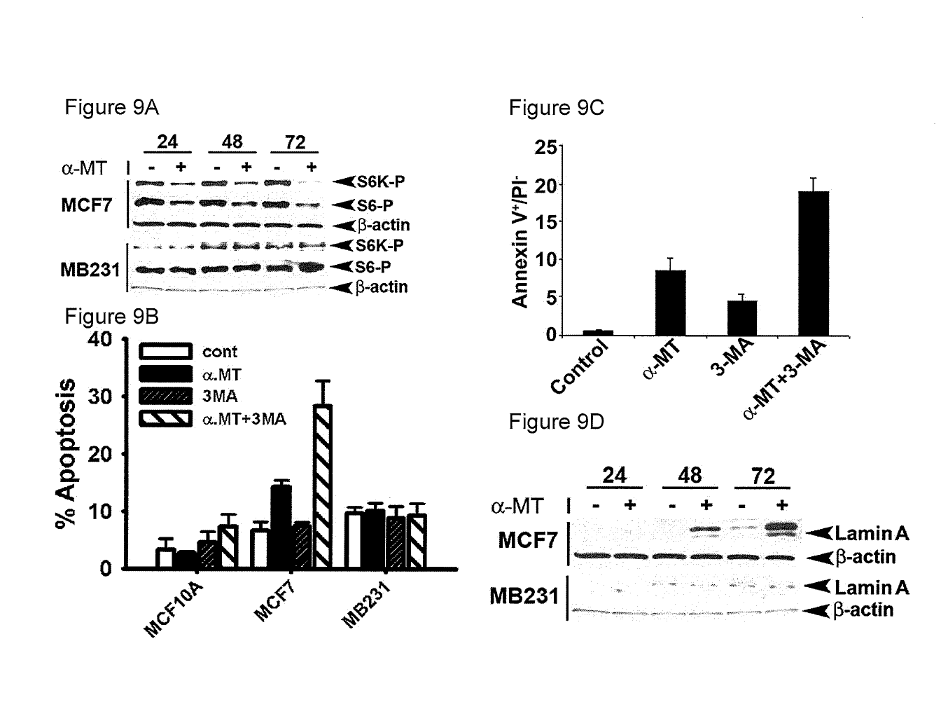

There was also evidence of mTOR inhibition in MCF7 cells when treated with α-MT as evident from the decreased phosphorylation of S6 and S6 kinase, the downstream targets of mTOR (FIG. 9A). Again, mTOR activity in SLC6A14-negative cells was not affected by α-MT. When autophagy was blocked with 3-MA, there was a marked induction of cell death in MCF7 cells in the presence of α-MT as monitored by cell cycle analysis with propidium iodide labeling (FIG. 9B). Treatment with α-MT alone caused significant cell death in MCF7 cells, and this effect was further potentiated by cotreatment with 3-MA. There was very little, if any, effect on cell death in MB231 cells and MCF10A cells in response to treatment with α-MT and 3-MA, either alone or together. Treatment of MCF7 cells with α-MT also induced apoptotic cell death as monitored by annexin V labeling (FIG. 9C). 3-MA also caused significant apoptosis, but when combined with α-MT, the effect was much greater than when the cells were treated with either agent alone. The apoptotic cell death caused in MCF7 cells by treatment with α-MT was also demonstrable by the cleavage of Lamin A (a specific substrate for activated caspase-6) (FIG. 9D). There was no evidence of apoptosis in SLC6A14-negative MB321 cells under identical conditions.

The influence of α-MT (2.5 mM) on cell migration and invasion was examined not with MCF7 cells but with ZR75.1 cells because MCF7 cells are non-invasive. α-MT did not have any significant influence on cell migration or invasion in ZR75.1 cells (migration in α-MT-treated cells was 87±7% of control, p>0.05; invasion in α-MT-treated cells was 95±14% of control, p>0.05).

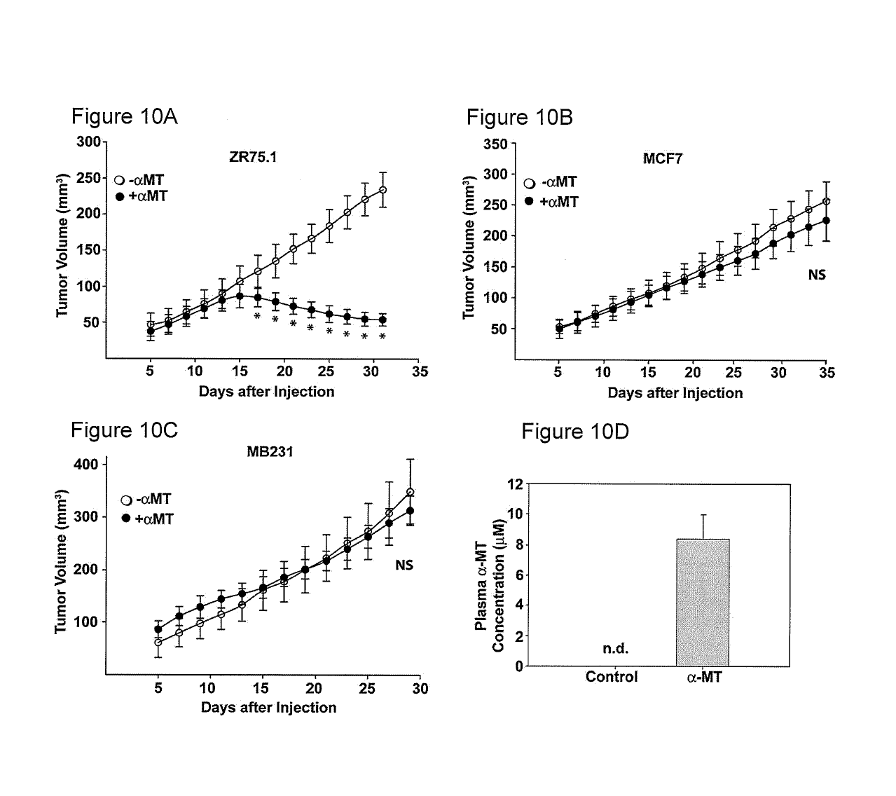

Inhibition of tumor growth by α-MT in vivo. To determine the therapeutic potential of SLC6A14 as a drug target for the treatment of ER-positive breast cancer in vivo, mouse xenograft studies were performed with two ER-positive breast cancer cell lines (ZR75.1 and MCF7) and one ER-negative breast cancer cell line (MB231). MCF7 cells did not form tumors unless estrogen was administered to the mice. On the other hand, ZR75.1 and MB231 cells readily formed tumors without the need for estrogen administration. α-MT in drinking water (2 mg/ml) reduced the growth of ZR75.1 cells (FIG. 10A), but did not affect the growth of MCF7 cells (in the presence of estrogen administration) or MB231 cells (FIG. 10B, C). The plasma concentration of α-MT in mice after two weeks of administration in drinking water (2 mg/ml) was 8.5±0.5 M (FIG. 10D). α-MT was not detectable in control mice.

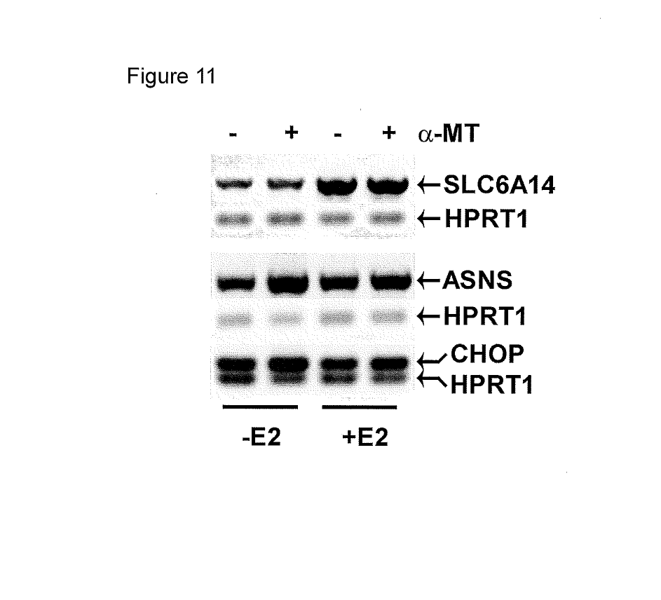

MCF7 cells, which are SLC6A14-positive, were not affected by α-MT in vivo even though the growth of these cells was inhibited by α-MT in vitro. MCF7 cells do not grow into a tumor in nude mice unless estrogen is administered; under these conditions, estrogen-induced upregulation of SLC6A14 most likely compensates for the blockade of the transporter by α-MT. This was confirmed in vitro. When MCF7 cells were grown for 24 h in phenol red-free culture medium in the absence and presence of estradiol (10 nM), the expression of SLC6A14 was higher in estradiol-treated cells (FIG. 11). In control cells cultured in the absence of estradiol, treatment with α-MT (2.5 mM, 24 h) induced the expression of asparagine synthetase and CHOP. The magnitude of this effect was significantly reduced in cells treated with estradiol (FIG. 11), showing that the elevated expression of SLC6A14 suppresses the efficacy of α-MT to block the transporter. Therefore, it may be necessary to optimize the treatment protocol (dose and treatment time) to demonstrate the efficacy of α-MT in blocking the growth of MCF7-derived tumors in mouse xenografts in the presence of exogenous estrogen.

Taken collectively, the results presented here provide evidence for SLC6A14 as an effective drug target for treatment of ER-positive breast cancer in vivo. These results can be summarized as follows. First, this example demonstrated the specific upregulation of SLC6A14 in ER-positive primary breast cancer specimens. Second, this example showed that treatment of ER-positive breast cancer cells with α-MT led to amino acid deprivation and autophagy and that cotreatment with an autophagy inhibitor caused apoptosis and cell death. Importantly, these effects were not seen in ER-negative breast cancer cells and in non-malignant mammary epithelial cells. Third, this example showed that, in a mouse xenograft model, administration of α-MT in drinking water was effective in reducing the tumor growth of ER-positive breast cancer cells with no effect on the tumor growth of ER-negative breast cancer cells. These studies identify a novel approach for the treatment of ER-positive breast cancer by targeting, for the first time, an essential amino acid transporter. α-MT is a potent blocker of SLC6A14 in vivo with its therapeutic efficacy evident at plasma concentrations in the micromolar range. Blockade of the transporter is unlikely to have off-target detrimental effects on normal tissues because the observed effects are specific to SLC6A14-positive tumor cells.

In conclusion, this example demonstrates that SLC6A14 is a potential drug target for the treatment of ER-positive breast cancer. α-MT in itself represents a viable drug for this purpose. It could also serve as a lead compound for the design and development of more potent blockers of SLC6A14 for future use. Therapeutic strategies are currently available for the treatment of ER-positive breast cancer using antiestrogens such as tamoxifen, but development of resistance to this mode of treatment has become a significant problem for successful therapy by this approach. Therefore, new therapeutic strategies with molecular targets that differ mechanistically from antiestrogens have great potential in breast cancer treatment. This example identifies SLC6A14 as one such target that needs to be investigated in humans for its potential in the treatment of ER-positive breast cancer.

This example has also published as Karunakaran et al., J Biol. Chem. 2011 Sep. 9; 286(36):31830-8. doi: 10.1074/jbc.M111.229518. Epub 2011 Jul. 19; “SLC6A14 (ATB0,+) protein, a highly concentrative and broad specific amino acid transporter, is a novel and effective drug target for treatment of estrogen receptor-positive breast cancer,” which is herein incorporated by reference.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims. All headings are for the convenience of the reader and should not be used to limit the meaning of the text that follows the heading, unless so specified.

### SEQUENCE LISTING FREE TEXT

SEQ ID NOs:1-14 <synthetic oligonucleotide primers>

What is claimed is:

1. A method of treating estrogen receptor positive breast cancer in a subject, the method comprising administering to the subject a composition comprising an inhibitor of the ATB^{0,+} amino acid transporter.

2. A method of killing estrogen receptor positive breast cancer cells, the method comprising contacting the cells with a composition comprising an inhibitor of the ATB^{0,+} amino acid transporter.

3. A method of inducing amino acid deprivation and/or autophagy in a cancer cell, the method comprising contacting the cancer cell with an inhibitor of the ATB^{0, +} amino acid transporter.

4. A method of inducing apoptosis in a cancer cell, the method comprising contacting the cancer cell with an inhibitor of the ATB^{0,+} amino acid transporter.

5. The method of inducing apoptosis in a cancer cell of claim 4, the method further comprising contacting the cancer cell with an inhibitor of autophagy.

6. The method of claim 5, wherein the inhibitor of autophagy is selected from 3-methyladenine, suppressive miR-101, lucanthone, chloroquine, hydroxychloroquine, N-acetyl-L-cysteine, L-asparagine, bafilomycin A1 from Streptomyces griseus, catalase, JRF 12 N2,N4-dibenzylquinazoline-2,4-diamine (DbeQ), (2S,3S)-trans-Epoxysuccinyl-L-leucylamido-3-methylbutane ethyl ester EST (E-64d), leupeptin hemisulfate, 2-(4-Morpholinyl)-8-phenyl-1(4H)-benzopyran-4-one hydrochloride (LY-294,002), pepstatin A, thapsigargin, or wortmannin from Penicillium funiculosum.

7. The method of 3, wherein the cancer is a breast cancer.

8. The method of claim 7, wherein the breast cancer is an estrogen receptor positive breast cancer.

9. The method of claim 1, wherein the inhibitor of the ATB^{0,+} amino acid transporter comprises a tryptophan derivative.

10. The method of claim 1, wherein the inhibitor of the ATB^{0,+} amino acid transporter comprises alpha methyl tryptophan.

11. The method of claim 1, wherein the composition comprises the L isomer of alpha methyl tryptophan and does not comprise the D isomer of alpha methyl tryptophan.

12. The method of claim 1, further comprising administration of an inhibitor of autophagy.

13. The method of claim 12, wherein the inhibitor of autophagy is selected from 3-methyladenine, suppressive miR-101, lucanthone, chloroquine, hydroxychloroquine, N-acetyl-L-cysteine, L-asparagine, bafilomycin A1 from Streptomyces griseus, catalase, JRF 12 N2,N4-dibenzylquinazoline-2,4-diamine (DbeQ), (2S,3S)-trans-Epoxysuccinyl-L-leucylamido-3-methylbutane ethyl ester EST (E-64d), leupeptin hemisulfate, 2-(4-Morpholinyl)-8-phenyl-1(4H)-benzopyran-4-one hydrochloride (LY-294,002), pepstatin A, thapsigargin, or wortmannin from Penicillium funiculosum.

14. The method of claim 1, further comprising administration of an additional therapeutic agent.

15. The method of claim 1, wherein the composition is formulated for parenteral delivery or enteral delivery.


SEQUENCE LISTING (14 sequences)

SEQ ID NO: 1 - DNA, 27 bp, artificial
  synthetic oligonucleotide primer
gtcgaccctgccttcaaagaactggta

SEQ ID NO: 2 - DNA, 25 bp, artificial
  synthetic oligonucleotide primer
aagcttgcactctcccctgttccta

SEQ ID NO: 3 - DNA, 20 bp, artificial
  synthetic oligonucleotide primer
gcacgccctctatgacaatg

SEQ ID NO: 4 - DNA, 18 bp, artificial
  synthetic oligonucleotide primer
ctcactctcctcggcttt

SEQ ID NO: 5 - DNA, 20 bp, artificial
  synthetic oligonucleotide primer
gagaaccaggaaacggaaac

SEQ ID NO: 6 - DNA, 19 bp, artificial
  synthetic oligonucleotide primer
gcagattcaccattcggtc

SEQ ID NO: 7 - DNA, 22 bp, artificial
  synthetic oligonucleotide primer
gaaggagaaagtgtcggcttca

SEQ ID NO: 8 - DNA, 22 bp, artificial
  synthetic oligonucleotide primer
taccaccttgccagacgatttg

SEQ ID NO: 9 - DNA, 19 bp, artificial
  synthetic oligonucleotide primer
ggctccgcaaatgctacga

SEQ ID NO: 10 - DNA, 21 bp, artificial
  synthetic oligonucleotide primer
cgccagacgagaccaatcatc

SEQ ID NO: 11 - DNA, 20 bp, artificial
  synthetic oligonucleotide primer
tggacctgacctgccgtcta

SEQ ID NO: 12 - DNA, 20 bp, artificial
  synthetic oligonucleotide primer
aggagtgggtgtcgctgttg

SEQ ID NO: 13 - DNA, 21 bp, artificial
  synthetic oligonucleotide primer
gcgtcgttagcgatgatgaac

SEQ ID NO: 14 - DNA, 24 bp, artificial
  synthetic oligonucleotide primer
cctcccatctccttcatgacatct